(19) United States
(12) Patent Application Publication
(10) Pub. No.: US 2026/0027015 A1
(43) Pub. Date: Jan. 29, 2026

(54) DENTAL SELF-HEALING ROOT CANAL FILLING COMPOSITION AND METHOD FOR MANUFACTURING THEREOF

(71) Applicant: Jong ho Song, Seoul (KR)

(72) Inventor: Jong ho Song, Seoul (KR)

(21) Appl. No.: 19/270,366

(22) Filed: Jul. 15, 2025

(30) Foreign Application Priority Data
Jul. 23, 2024 (KR) 10-2024-0096796

(51) Int. Cl. A61K 6/54; A61K 6/70; A61K 6/79

(52) U.S. Cl. CPC A61K 6/54; A61K 6/70; A61K 6/79

(57) ABSTRACT

Disclosed are dental self-healing root canal filling compositions and methods of preparing the same.

The compositions comprise a hardener, a self-healing promoter, a calcium solubilization inhibitor, a bioactive agent, a curing aid, a radiopaque material, a thickener, and purified water.

The compositions are capable of minimizing microcracks, improving tissue density, and providing self-healing properties.

### BACKGROUND

### 1. Field

The present invention relates to a dental self-healing root canal filling composition and a method for manufacturing the same, and more particularly, to a biocompatible dental self-healing root canal filling composition and a method for manufacturing the same, in which a pozzolanic reactant (nano-silica, silica fume, or fumed silica) is added to a tricalcium silicate-based composition free of aluminum components, so that cracks in the tooth can be self-healed.

### 2. Description of the Related Art

Root canal filling materials are used to remove inflammation from the affected area when apical disease is caused by inflammation of the pulp tissue, and to fill the root canal in three dimensions to seal the root canal system, with the aim of promoting healing of the periapical tissues.

Root canal filling materials should satisfy various characteristics, including sufficient setting time, adhesive capability, radiopacity, prevention of discoloration of remaining tooth structure, solubility in solvents for easy removal, excellent sealing ability, suppression of tissue damage or cell necrosis upon contact with biological tissues, insolubility in saliva and tissue fluids, and inhibition of bacterial growth.

Various materials such as zinc oxide-eugenol, zinc oxide-fatty acid, salicylate, glass ionomer, silicone, epoxy resin, methacrylate resin, and mineral trioxide aggregate (MTA) have been developed.

In particular, mineral trioxide aggregate (MTA) is a material that hardens upon reaction with water and increases in strength over time. It is biocompatible, integrates well with pulp and periapical tissues, and is known not only to provide excellent sealing ability that prevents bacterial infiltration into the root canal, but also to protect the pulp when applied to exposed pulp areas.

However, due to the need for rapid setting conditions of MTA during root canal treatment, cement-based compositions containing aluminum components have been additionally used to reduce the setting time. Although aluminum ions leached from such aluminum-containing cement compositions are known to be substances that can cause diseases such as rheumatoid arthritis and Alzheimer's disease, they are still in use in practice.

In addition, MTA may develop microcracks during the setting process due to shrinkage, expansion, or the leaching of alkaline components. Such microcracks not only reduce mechanical strength but also provide spaces where harmful bacteria can reside and proliferate, potentially leading to inflammation.

Therefore, to solve the above problems, there is a need to develop a root canal filling material composition based on tricalcium silicate without aluminate components and a root canal filling material with a self-healing function that repairs itself in the event of microcracks.

### SUMMARY

The present invention has been conceived in consideration of the above points, and its purpose is to provide a dental self-healing root canal filling composition and a method for preparing the same, which comprises a self-healing promoting agent, a calcium solubilization inhibitor, a bioactive agent, a curing aid, a radiopaque material, a thickener, and purified water as a hardener based on lithium calcium silicate, which has a self-healing function of optimizing strength and repairing microcracks.

To accomplish the above objectives, the present invention provides a dental self-healing root canal filling composition that addresses the limitations of conventional materials by offering enhanced biocompatibility, sealing performance, and self-healing capability. The composition comprises a synergistic blend of components including a hardener, a self-healing promoter, a calcium solubilization inhibitor, a bioactive agent, a curing aid, a radiopaque material, a thickener, and purified water.

In one aspect, the composition comprises 25 to 35 wt % of a hardener, 1 to 10 wt % of a self-healing promoter, 0.5 to 1.5 wt % of a calcium solubilization inhibitor, 5 to 10 wt % of a bioactive agent, 0.3 to 1 wt % of a curing aid, 25 to 35 wt % of a radiopaque material, 15 to 40 wt % of a thickener, and 0.5 to 2 wt % of purified water.

In a preferred embodiment, the hardener includes tricalcium silicate (C₃S) with a purity of 90 to 98% and an average particle size of 3 μm or less. The self-healing promoter comprises one or more materials selected from the group consisting of nano-silica, silica fume, and fumed silica, preferably having a particle size of 100 nm or less and an average particle size between 10 and 20 nm.

The calcium solubilization inhibitor comprises sodium fluoride (NaF), while the bioactive agent comprises lithium carbonate (Li₂CO₃). The curing aid may include one or more compounds selected from calcium sulfate hemihydrate, calcium sulfate dihydrate, calcium chloride, and calcium formate. The radiopaque material may include one or more selected from zirconium oxide, tantalum oxide, zinc oxide, barium sulfate, bismuth oxide, barium oxide, iodoform, and calcium tungstate. The thickener may comprise polyethylene glycol, 1,3-propanediol, or 1,3-butanediol.

The composition exhibits desirable physical properties for clinical use, including a compressive strength in the range of 5 to 10 MPa and a flow rate of 25 to 30 mm, and is suitable for use in self-healing root canal procedures.

In another aspect, the invention provides a method for preparing the dental self-healing root canal filling composition. The method includes the steps of preparing a hardener comprising tricalcium silicate; mixing the functional components within the prescribed weight ratios; and subjecting the resulting mixture to vacuum treatment at −0.1 MPa or lower for 15 to 20 minutes to remove entrapped air and improve the filling density.

In a preferred manufacturing method for the hardener, tricalcium silicate is prepared by mixing calcium carbonate and silica in a 3:1 molar ratio with 250 to 350 cc of water, followed by ball milling, mold drying, calcination at 1,350 to 1,600° C., quenching, and final grinding and sieving to achieve a particle size of 3 μm or less.

This invention provides an improved, biologically safe root canal filling composition capable of sealing and self-healing, offering enhanced clinical outcomes in endodontic procedures.

A dental self-healing root canal filling composition according to the present invention is free from toxic aluminum, which is known to contribute to Alzheimer's disease and exacerbate symptoms in patients with rheumatoid conditions. The composition exhibits a self-healing functionality wherein Si⁺ ions are reactivated at post-curing crack sites to facilitate autonomous repair of the cracks. Additionally, Li⁺ ions are reactivated at these crack sites to provide a sustained antimicrobial effect. The inclusion of lithium-based compounds further enhances the reduction of periapical lesions, improves dimensional stability, and imparts sufficient mechanical strength to allow for retreatment procedures following curing.

Furthermore, the method for preparing the dental self-healing root canal filling composition according to the present invention includes a self-healing promoter comprising nano-silica, silica fume, and/or fumed silica, thereby enabling a simple and efficient manufacturing process.

### BRIEF DESCRIPTION OF DRAWINGS

FIG. 1 illustrates the manufacturing process of a root canal filling material according to an embodiment of the present invention.

FIG. 2 is an SEM photograph of a root canal filling material after curing according to an embodiment of the present invention.

FIG. 3 is an XRD analysis of tricalcium silicate according to an embodiment of the present invention.

FIG. 4 shows a cure time test of a root canal filling material according to an embodiment of the present invention.

FIG. 5 shows a compressive strength test of a root canal filling material according to an embodiment of the present invention.

FIG. 6 shows a self-healing test of a root canal filling material according to an embodiment of the present invention.

FIG. 7 shows a flowability test of a root canal filling material according to an embodiment of the present invention.

### DETAILED DESCRIPTION

All terms used in this specification have been chosen in their current common usage, taking into account the features of the invention, but they may vary according to the intentions, conventions, or practices of those of ordinary skill in the art, or the appearance of new technologies. In addition, wherever the inventor has specified a term in this invention, the meaning of the term will be stated in the description of the invention. Therefore, the terms used in this invention should be interpreted based on the actual meaning of the term and the overall description of the invention, rather than the mere designation of the term.

Referring now to the accompanying drawings, a dental self-healing root canal filling composition according to an embodiment of the present invention and a method for preparing the same will be described in detail.

The present invention provides a dental self-healing root canal filling composition.

A dental self-healing root canal filling composition according to an embodiment of the present invention comprises a hardener, a self-healing promoter, a calcium solubilization inhibitor, a bioactive agent, a curing aid, a radiopaque material, a thickener, and purified water. It may also include 25 to 35 wt % of a hardener, 1 to 10 wt % of a self-healing promoter, 0.5 to 1.5 wt % of a calcium solubilization inhibitor, 5 to 10 wt % of a bioactive agent, 0.3 to 1 wt % of a curing aid, 25 to 35 wt % of a radiopaque material, 15 to 40 wt % of a thickener, and 0.5 to 2 wt % of purified water.

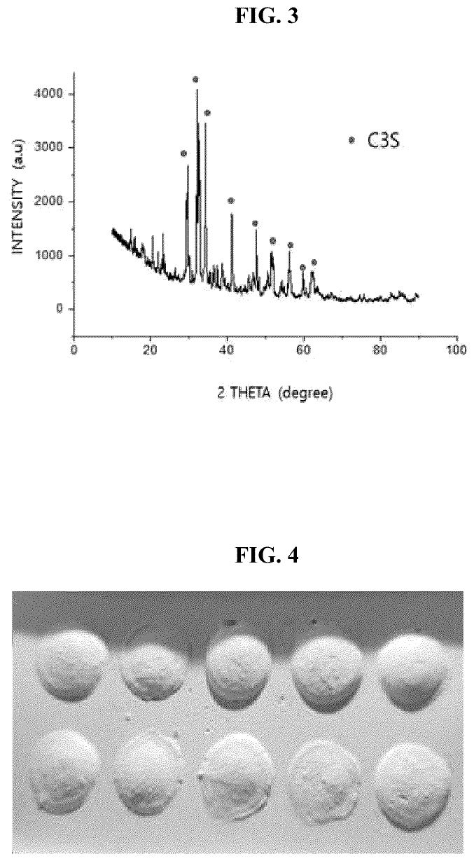

Referring to FIG. 3, the hardener may comprise tricalcium silicate (C₃S). The hardener may comprise 25 to 35 wt % by weight of the composition. The tricalcium silicate may be prepared by reacting a mixture comprising calcium carbonate and silica with a heat treatment, followed by quenching. The tricalcium silicate may be cured by reacting with water to form a hydrate as a white, free-flowing powder.

The tricalcium silicate may therefore have cementing properties, thus minimizing the loss of composition that may occur in the apical region of the tooth due to near-zero water partitioning, thereby minimizing the loss of strength due to reduced density. Furthermore, the tricalcium silicate may affect the setting time, which can be an important factor in endodontic treatment, as a delayed setting time may result in the inability to completely seal the space inside the root canal.

Regarding strength, the post-curing strength of the dental root canal filling material of the present invention can be determined by tricalcium silicate. Therefore, it can be determined by the ratio of purified water to tricalcium silicate (W/C ratio). The ratio of purified water to tricalcium silicate may depend on the amount of tricalcium silicate formulation and particle size. Thus, the filling material prepared with the dental root canal filling composition of the present invention can have a compressive strength of 5 to 10 MPa that can be removed from the tooth if necessary by adjusting the water content.

In addition, regarding setting time, calcium hydroxide formed from tricalcium silicate is more than three times more abundant than that formed from other cements. Therefore, a high content of tricalcium silicate is characterized by a faster setting time and increased initial strength. Therefore, it is necessary to optimize the setting time and the root canal filling condition required during endodontic treatment of teeth. If the setting time is too short, the filling material may become hard and difficult to fill in the root canal, and if it is too long, the root canal may be contaminated by leachate or blood from the surrounding environment.

The following Chemical Formula 1 represents the hydration reaction of tricalcium silicate.

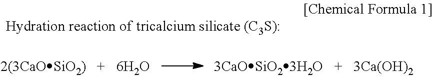

Thus, the hardener tricalcium silicate may comprise 25 to 35 wt % by weight of the composition. If the content of tricalcium silicate is less than 25 wt %, the compressive strength may be reduced and the setting time may be prolonged, resulting in changes in properties, and if the content is greater than 35 wt %, the fluidity may be reduced, making it unsuitable as a micro root canal filling material, and the compressive strength may be increased, making it difficult to remove if necessary. Also, the tricalcium silicate having a purity of 90 to 98% and an average particle size of 3 μm or less can be used. If the average particle size is greater than 3 μm, it may not be dense and the strength may be reduced.

A self-healing promoter is included in the present invention. The self-healing promoter may comprise from 1 to 10 wt % based on the total weight of the composition. The self-healing promoter may include at least one of nano-silica (SiO₂), silica fume (SiO₂) and fumed silica (SiO₂). Furthermore, the self-healing promoter may be selected to have a particle size of 100 nm or less, with an average particle size of 10 to 20 nm. As another example, the self-healing promoter may have a particle size of 50 nm or less, with an average particle size between 5 and 10 nm.

Self-healing refers to the process in which microcracks caused by shrinkage, expansion, or ionic elution during the curing of the filling material, or microcracks subsequently caused by external forces after normal filling, are filled with bonding material, thereby repairing the microcracks. As a result of this self-healing, it may not provide a space for bacteria and other microorganisms to proliferate. The aforementioned pozzolanic reactants-nano-silica, silica fume, or fumed silica with a particle size of 100 nm or less-possess hydrophilic surface modifications that significantly enhance the adsorption of surrounding ions. This accelerates the crystallization process with mineral components such as calcium and phosphorus, resulting in strong binding forces that improve the self-healing effect.

In other words, the filling material of the present invention may improve its bonding with the surrounding tissue, thereby effectively filling and sealing the space inside the root canal. The nano-silica, silica fume, or fumed silica can also function as a bonding enhancer. The nano-silica, silica fume, or fumed silica can improve the dimensional stability of the filling material during the curing process, minimizing shrinkage and enhancing the mechanical properties of the filling material by strengthening the bonding force of the filling material. In addition, when shear stress is applied externally, the viscosity decreases, and in the absence of shear stress, the network bonds of the silica are restored and the fluidity is reduced, thus facilitating viscosity control.

The nano-silica is a non-toxic, odorless, non-polluting inorganic chemical material synthesized by a pozzolanic reaction with nanometer-sized particles and controlled microstructure at the nanometer level. The nano-silica exhibits a particle size of 15 to 20 nm, a density of 0.128 to 0.141 g/cm³ and a specific surface area of 559 to 685 m²/g. In general, nano-silica is characterized by having countless mesopores in the matrix, and in addition to the advantage of improving thermal insulation performance, it exhibits high mechanical properties when mixed with cementitious materials (C₃S). The nano-silica can be applied in various fields such as coatings, adhesives, rubber products, plastics, textiles, antibacterial agents, and catalysts, but in the present invention, it can be used for self-healing by filling microcracks.

The silica fume can be obtained by collecting waste gases from the manufacture of silicon alloys such as silicon or ferrosilicon. The silica fume can exhibit a void filling effect from the beginning of the hydration of tricalcium silicate (C₃S) through the pozzolanic reaction. Therefore, in the present invention, this action can be utilized to fill the microcracks to be used for self-healing.

The fumed silica consists of 12 nm primary particles that form agglomerates interconnected in a three-dimensional branched structure, resulting in the formation of multiple mesopores. The density and specific surface area of the fumed silica are approximately 0.05 g/cm³ and 200 m²/g, respectively. The particle size of the fumed silica ranges from 5 to 24 nm, which is approximately 4 to 20 times smaller than that of silica fume, which has an average particle size of 0.1 μm. The fumed silica may be prepared in powder form with either hydrophilic or hydrophobic properties. It can undergo a pozzolanic reaction with calcium hydroxide [Ca(OH)₂], a hydration product of tricalcium silicate, to form insoluble calcium silicate hydrate (C—S—H gel), thereby further densifying the internal matrix. This property enables the fumed silica to be effectively utilized in the present invention for filling microcracks.

Thus, at least one of the nano-silica, silica fume, and fumed silica may comprise 1 to 10 wt % by weight of the composition. If the content is less than 1 wt %, it may be difficult for the self-healing function of the filler of the present invention to be exerted and the mechanical property, strength, may be reduced. In addition, if the content is more than 10 wt %, it may be difficult to use the inventive filler due to excessively low flowability.

The calcium solubilization inhibitor is included in the present invention. The calcium solubilization inhibitor may comprise 0.5 to 1.5 wt % based on the total weight % of the composition. Furthermore, the calcium solubilization inhibitor may comprise sodium fluoride (NaF). The calcium hydroxide formed from calcium silicate may have a higher solubility of Ca⁺ ions as the amount increases, and the higher solubility may lead to an increase in porosity, which may ultimately reduce the strength of the root canal filling material. The calcium solubilization inhibitor, sodium fluoride, can react with the initially dissolved calcium ions to form insoluble CaF₂ and form a protective film, thereby inhibiting the dissolution of calcium from the inside. In addition, when reacting with aqueous solutions, sodium fluoride can release fluoride to provide dental hard tissue and caries prevention and pulp protection.

Thus, the sodium fluoride as the calcium solubilization inhibitor may comprise 0.5 to 1.5 wt % based on the total weight of the composition. If the content of the sodium fluoride is less than 0.5 wt %, the dissolution of calcium cannot be prevented, and it may be difficult to prevent pain due to inflammation caused by bacteria. In addition, if the content of the sodium fluoride is more than 1.5 wt %, it may cause vomiting, etc. due to excessive addition.

A bioactive agent is included in the present invention. The bioactive agent may comprise 5 to 10 wt % based on the total weight of the composition. The bioactive agent may comprise lithium carbonate (Li₂CO₃). Lithium carbonate enhances biocompatibility by reducing cytotoxicity and exhibiting antimicrobial activity, while also contributing to the reduction of root lesions and providing pulp-protective effects. Additionally, upon contact with water, dissolved lithium ions and carbon dioxide (CO₂) gas may recombine and recrystallize into lithium carbonate (Li₂CO₃), which can accumulate in micropores or cracks, thereby facilitating the self-healing of such cracks.

Thus, the lithium carbonate as the bioactive agent may comprise 5 to 10 wt % by weight of the composition. If the content of lithium carbonate is less than 5 wt %, the compressive strength may be reduced or the flowability may be reduced. Also, if the lithium carbonate content is greater than 10 wt %, excessive addition may cause symptoms of lithium poisoning such as vomiting.

The curing aid is included in the present invention. The curing aid may be present in an amount ranging from 0.3 to 1 wt % based on the total weight of the composition. The curing aid may comprise at least one selected from calcium sulfate hemihydrate (CaSO₄·½H₂O), calcium sulfate dihydrate (CaSO₄·2H₂O), calcium chloride (CaCl₂), and calcium formate [Ca(HCOO)₂]. In a preferred embodiment, calcium chloride is selected as the curing aid. Calcium chloride can shorten the setting time by promoting the hydration reaction of tricalcium silicate. Specifically, when tricalcium silicate particles are exposed to moisture, they undergo hydration to form calcium silicate hydrate (C—S—H), which creates a coating layer around the particles. As hydration progresses, this C—S—H layer thickens, slowing further moisture penetration and consequently reducing the rate of the hydration reaction, thereby delaying the setting time. However, when an aqueous solution of calcium chloride is added, chloride ions diffuse into the C—S—H layer, disrupting its structure and allowing moisture to penetrate more easily into the particle core. This accelerates the hydration reaction and consequently shortens the setting time.

In addition, an aqueous solution of calcium chloride can help to improve solubility and prevent foaming. In the case of the dental root canal filling material of the present invention, in order to minimize solubility and bubbling after curing, the water to tricalcium silicate (W/C) ratio, the radiopaque material content, and the molecular weight of the thickener can be considered. In particular, in the present invention, the solubility and porosity can be minimized by controlling the ratio of W/C while using polyethylene glycol as a thickener and maximizing the radiopaque material content. In other words, the ratio of W/C contained in the dental root canal filling material according to an embodiment of the present invention can affect the increase or decrease of solubility and porosity.

Thus, a higher ratio of W/C may result in a higher solubility of Ca⁺ ions due to an increase in calcium hydroxide, which is the hydrated material in the dental root canal filling material of the present invention. This increase in solubility may lead to an increase in pores, which may ultimately result in lower strength. Therefore, in embodiments of the present invention, the content of water in the dental root canal filling material can be adjusted to the content of the aqueous solution of calcium chloride to minimize solubility and foaming.

Accordingly, the curing aid may comprise 0.3 to 1 wt % based on the total weight of the composition. On the other hand, if the content of calcium chloride is less than 0.3 wt %, the setting time is prolonged, and the dental root canal filling material of the present invention may be washed away by the bloodstream, and the volumetric stability may be reduced. If the calcium chloride content is greater than 1 wt %, the structure of the cured material may exhibit porosity.

A radiopaque material is included in the present invention. The radiopaque material may comprise 25 to 35 wt % of the composition and may include at least one selected from zirconium oxide (ZrO₂), tantalum oxide (Ta₂O₅), zinc oxide (ZnO), barium sulfate (BaSO₄), bismuth oxide (Bi₂O₃), barium oxide (BaO), iodoform (CHI₃), and calcium tungstate (CaWO₄). In a preferred embodiment, zirconium oxide may be selected as the radiopaque material. The radiopaque material may be included as an inorganic compound to enhance radiographic contrast, allowing for improved visualization of treatment prognosis following root canal therapy. Furthermore, the content of the radiopaque material may influence the solubility and help minimize bubble formation after curing of the filling material.

Thus, the zirconium oxide as the radiopaque material may comprise 25 to 35 wt % by weight of the composition. If the content of the zirconium oxide is out of the range, it may be difficult to improve the radiographic contrast during radiography to observe the prognosis of the treatment.

A thickener is included in the present invention. The thickener may comprise from 15 to 40 wt % of the composition and may include one or more of polyethylene glycol (C₂ₙH₄ₙ₊₂Oₙ₊₁), 1,3-propanediol (CH₂(CH₂OH)₂), and 1,3-butanediol (C₄H₁₀O₂). In a preferred embodiment, polyethylene glycol, preferably PEG-300, may be selected as the thickener. The thickener may be included to control the flowability of the composition. Furthermore, the molecular weight of the thickener may influence its solubility and contribute to minimizing bubble formation after curing the filler composition of the present invention.

Thus, the thickener, polyethylene glycol (PEG-300), may comprise from 15 to 40 wt % by weight of the composition. If the content of the polyethylene glycol is less than 15 wt %, the composition may lack sufficient flowability and self-healing ability. If the polyethylene glycol content is greater than 40 wt %, excessive fluidity may result in injury to the fascia tissue from exposure to tricalcium silicate.

The purified water is included in the present invention. The purified water may comprise 0.5 to 2 wt % by weight of the composition. The purified water can be optimally blended with the tricalcium silicate to minimize the post-curing solubility and bubbling of the filler of the invention. Therefore, if the content of the purified water is out of the range, the strength of the present invention may be excessive and may not be easy to remove during treatment, and if the strength is reduced, it may cause cracking due to external impact, which may cause inflammation caused by bacteria.

Accordingly, the dental root canal filling compositions of the present invention can form an apatite group having a chemical formula of Me₁₀(PO₄)₆X₂, wherein Me represents one of calcium (Ca), barium (Ba), or strontium (Sr), and X represents at least one selected from hydroxyl (—OH), fluoride (F), or chloride (Cl). Examples of apatite compounds that may be formed include calcium hydroxyapatite [Ca₁₀(PO₄)₆(OH)₂], barium hydroxyapatite [Ba₁₀(PO₄)₆(OH)₂], strontium hydroxyapatite [Sr₁₀(PO₄)₆(OH)₂], calcium fluorapatite [Ca₁₀(PO₄)₆F₂], barium fluorapatite [Ba₁₀(PO₄)₆F₂], strontium fluorapatite [Sr₁₀(PO₄)₆F₂], calcium chloroapatite [Ca₁₀(PO₄)₆Cl₂], barium chloroapatite [Ba₁₀(PO₄)₆Cl₂], and strontium chloroapatite [Sr₁₀(PO₄)₆Cl₂]. Furthermore, the dental root canal filling compositions of the present invention may possess sufficient mechanical strength to allow for removal during retreatment, minimize microcrack formation, and promote self-healing.

### Manufacturing Method

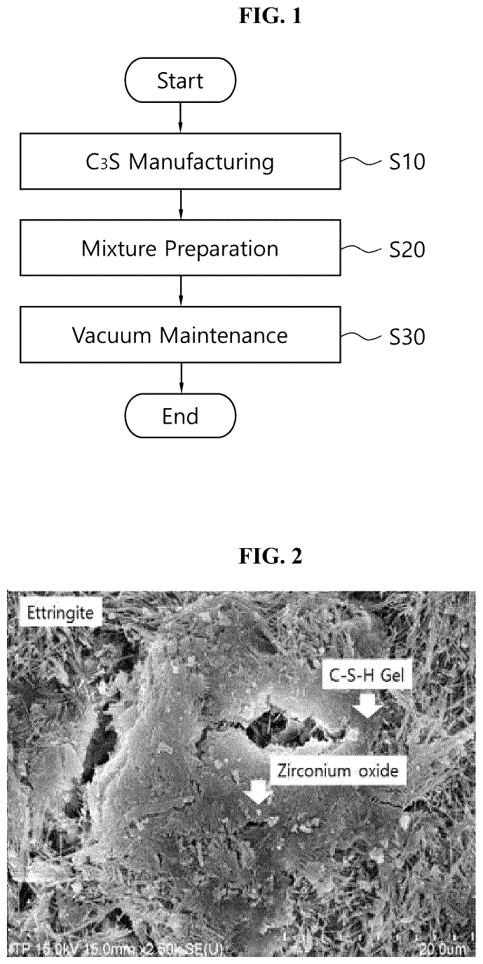

FIG. 1 illustrates the manufacturing process of a dental self-healing root canal filling material according to an embodiment of the present invention.

The present invention provides a method for preparing a dental self-healing root canal filling material.

A method for preparing a dental root canal filling material according to an embodiment of the present invention comprises: a first step S10 of preparing a hardener, tricalcium silicate (C₃S); a second step S30 of preparing a dental root canal filling material, comprising: tricalcium silicate (C₃S) 25 to 35 wt %; a self-healing promoter 1 to 10 wt %; a calcium solubilization inhibitor 0.5 to 1.5 wt %; bioactive agent 5 to 10 wt %; curing aid 0.3 to 1 wt %; radiopaque material 25 to 35 wt %; thickener 15 to 40 wt %; and purified water 0.5 to 2 wt %; and a third step S50 of maintaining the mixture under a vacuum of −0.1 MPa or less for 15 to 20 minutes to eliminate bubbles and increase the filling density, characterized in that the self-healing promoter comprises at least one of nano-silica, silica fume and fumed silica (SiO₂).

The following ingredients are described in the “Composition” section above and will not be described in detail.

The first step S10 is to prepare a hardener, tricalcium silicate (C₃S). The tricalcium silicate can block the loss of filling material that may occur in the apex region of the tooth due to near-zero water partitioning, thereby minimizing the loss of strength due to reduced density. Furthermore, the tricalcium silicate can influence the setting time, i.e., a delay in the setting time can lead to the problem of not being able to completely seal the space inside the root canal, so tricalcium silicate can be included in the present invention in an optimal content.

The above tricalcium silicate can be prepared by six processes:

First, using oxide raw materials of calcium carbonate (CaCO₃) and silica (SiO₂), the first mixture is prepared by mixing the calcium carbonate (CaCO₃):silica (SiO₂) in a molar ratio of 3:1 with 250 to 350 cc of water.

Second, the first mixture is placed in a zirconia container and mixed for 4 to 5 hours using a ball mill to prepare the second mixture.

Third, the second mixture is poured into a silicone mold and dried using a heating plate.

Fourth, the dried second mixture is heat-treated in an electric furnace at 1,350 to 1,600° C. for 5 to 6 hours to calcine the second mixture, followed by quenching. Preferably, the calcination temperature of the heat treatment furnace in the present invention may be 1,400 to 1,600° C. The temperature of 1,400 to 1,600° C. may be suitable for preparing tricalcium silicate with a purity of 90% or more. On the other hand, if the temperature is below the range, free lime (CaO) may be formed. This free lime structure may be undesirable as it may delay curing or may swell upon curing. In addition, tricalcium silicate with a purity of more than 99% can be produced above 1,600° C., but the manufacturing process is energy intensive and may be inefficient.

In addition, the calcination time of 5 to 6 hours can be carried out in two stages: at least 1 hour of the total 5 hours or more is for decarbonization of calcium carbonate at low temperatures, and less than 4 hours may be required for crystal phase formation at high temperatures. On the other hand, if the calcination time is below the range, the reaction time between the mixed materials is not sufficient, and the cement hardening may not be homogeneous due to unreacted residual materials.

Quenching can also be performed at a cooling rate of 200 to 300° C./min. The crystal structure at the firing temperature should be maintained during quenching. On the other hand, if the cooling rate is below the range, part of the tricalcium silicate may be converted to dicalcium silicate during the cooling process.

Fifth, mixing and grinding the quenched second mixture. The mixing and grinding may be carried out for 24 to 30 hours using a ball mill.

Sixth, the ground second mixture can be sieved to prepare a tricalcium silicate having an average particle size of 3 μm or less.

The second step S20 is to prepare a mixture. The mixture comprises, with respect to the total weight % of the dental root canal filling material, 25 to 35 wt % of tricalcium silicate (C₃S); 1 to 10 wt % of a self-healing promoter; 0.5 to 1.5 wt % of a calcium solubilization inhibitor; 5 to 10 wt % of a bioactive agent; 0.3 to 1 wt % of a curing aid; 25 to 35 wt % of a radiopaque material; 15 to 40 wt % of a thickener; and 0.5 to 2 wt % of purified water.

The tricalcium silicate may affect the setting time and strength of the root canal filling material of the present invention.

The self-healing promoter may comprise at least one of nano-silica, silica fume and fumed silica (SiO₂). At least one of the above nano-silica, silica fume, and fumed silica (SiO₂) has a hydrophilic surface modification during the curing process of the filler material, which has a large effect of adsorbing surrounding ions, so that the process of crystallizing into mineral components such as calcium and phosphorus is fast and the binding force is large, so that the self-healing effect can be greatly enhanced, and the dimensional stability can be improved to minimize shrinkage and improve the mechanical properties by strengthening the binding force of the filler material.

The calcium solubilization inhibitor may include sodium fluoride (NaF). Sodium fluoride can react with initially dissolved calcium ions to form insoluble calcium fluoride (CaF₂), thereby creating a protective film that inhibits further calcium dissolution from the interior. In addition, when reacting with an aqueous solution, sodium fluoride can release fluoride ions, which contribute to the reinforcement of dental hard tissues, prevention of dental caries, and protection of the dental pulp.

The bioactive agent may comprise lithium carbonate (Li₂CO₃). Lithium carbonate enhances biocompatibility by reducing cytotoxicity and providing antimicrobial effects, while also contributing to the reduction of root lesions and offering pulp protection. Furthermore, upon contact with moisture, the dissolved lithium ions and carbon dioxide (CO₂) gas may recrystallize into lithium carbonate (Li₂CO₃), which can accumulate in micropores or cracks, aiding in the self-healing of such defects.

The curing aid may comprise at least one selected from calcium sulfate hemihydrate (CaSO₄·½H₂O), calcium sulfate dihydrate (CaSO₄·2H₂O), calcium chloride (CaCl₂), and calcium formate [Ca(HCOO)₂]. The curing aid may accelerate the hydration reaction of tricalcium silicate, thereby reducing the setting time.

The radiopaque material may comprise at least one selected from tantalum oxide (Ta₂O₅), zinc oxide (ZnO), barium sulfate (BaSO₄), bismuth oxide (Bi₂O₃), barium oxide (BaO), iodoform (CHI₃), and calcium tungstate (CaWO₄). The radiopaque material may be incorporated into the composition as an inorganic additive to improve radiographic contrast during imaging, enabling observation of the treatment outcome following root canal procedures.

The thickener may comprise at least one selected from polyethylene glycol (C₂ₙH₄ₙ₊₂Oₙ₊₁), 1,3-propanediol (CH₂(CH₂OH)₂), and 1,3-butanediol (C₄H₁₀O₂). In a preferred embodiment, the thickener is polyethylene glycol, preferably PEG-300. The thickener may be included in the present invention to control the flowability of the composition.

The purified water can improve the strength of the present invention with an optimal formulation ratio with tricalcium silicate.

The third step S30 is to maintain the mixture under vacuum. The mixture can be maintained under a vacuum of −0.1 MPa or less for 15 to 20 minutes to eliminate bubbles and increase the filling density.

The dental root canal filling compositions prepared by the above process can be self-healing, minimize microcracks, be insoluble in water, have improved compressive strength, and have adequate flowability and film thickness.

### EXAMPLES

Examples and embodiments are described below. The following examples and embodiments are provided for purposes of illustrating the present disclosure only.

### Example 1

Using the oxide raw materials of calcium carbonate (CaCO₃) and silica (SiO₂), Mixture-1 was prepared by mixing 300 cc of water with a molar ratio of CaO:SiO₂ of 3:1. The above mixture-1 was placed in a zirconia container and mixed using a ball mill for 4 hours to prepare mixture-2. The homogenized mixture-2 was poured into a silicone mold of 50×60×20 (mm) and dried using a heating plate to prepare the bulk form. The dried bulk form of the above mixture-2 was placed in a square alumina crucible and calcined in an electric furnace at 1,400° C. for 5 hours. After calcination, the powder was taken out to room temperature and quenched. The quenched powdered mixture-2 at room temperature was placed in a zirconia container, mixed with a certain amount of ethyl alcohol, and mixed and ground for 24 hours using a ball mill. The ground mixture-2 was sieved to obtain tricalcium silicate powder with an average particle size of 3 μm. FIG. 3 is an XRD analysis of tricalcium silicate according to an example of the present invention.

### Example 2

Tricalcium silicate was prepared by the same method as in Example 1 except that the average particle size was 5 μm.

### Example and Comparison

### [Example 1]—Root Canal Fillings

The composition comprises 25 wt % of tricalcium silicate (C₃S) prepared according to Example 1, 7.2 wt % of lithium carbonate (Li₂CO₃), a bioactive agent, 0.5 wt % of sodium fluoride (NaF), a calcium solubilization inhibitor, 1.8 wt % of fumed silica (SiO₂), a self-healing promoter, calcium chloride (CaCl₂) 0.3 wt %, zirconium oxide (ZrO₂) 34.2 wt %, radiopaque material, polyethylene glycol-300 (PEG-300) 30 wt %, thickener, and purified water 1 wt % were mixed in a ball mill at 250 rpm for 1 hour to prepare the mixture. In order to remove bubbles and increase the filling density of the above mixture, the root canal filling material was prepared by maintaining it under a vacuum of −0.1 MPa or less for 20 minutes.

### [Example 2 and Comparative Example 1 and Comparative Example 7]—Root Canal Fillings

Table 1 below shows embodiments and comparative examples with different tricalcium silicate (C₃S) content and average particle size of the root canal filling material. Example 2 has an approximate median tricalcium silicate content, Comparative Example 1 has a tricalcium silicate content below the range, and Comparative Example 7 has a tricalcium silicate with an average particle size of 5 μm prepared according to Example 2. The root canal filling material was prepared in the same manner as Example 1 except that the other ingredients and contents were applied according to Table 1 below.

### Examples 3 Through 5 and Comparative Examples 2 Through 4

Table 2 below shows embodiments and comparative examples with different lithium carbonate (Li₂CO₃) content in the root canal filling material. In Examples 3 through 5, the minimum, median, and maximum values for the lithium carbonate (Li₂CO₃) content were applied, and in Comparative Examples 2 through 4, the lithium carbonate was applied as none, below range, or above range, and the root canal filling material was prepared in the same manner as in Example 1 except that the other ingredients and content were applied according to Table 2 below.

### Example 6 Through Example 10 and Comparative Example 5 and Comparative Example 6

Table 3 below compares embodiments with different ingredients (at least one of nano-silica, silica fume, and fumed silica) and content of the self-healing promoter in the root canal filling material. Embodiment 6 and Embodiment 7 applied a minimum and maximum value of fumed silica content, Embodiment 8 used a mixture of fumed silica and silica fume in a ratio of 1:1 instead of fumed silica as a self-healing promoter, and Embodiment 9 used a mixture of fumed silica and nano-silica in a ratio of 1:Example 9 used a mixture of fumed silica and nano-silica in a ratio of 1:1 instead of fumed silica as a self-healing promoter, Example 10 used a mixture of nano-silica and silica fume in a ratio of 1:1, Comparative Example 5 did not contain fumed silica, Comparative Example 6 applied an excess of the content range of fumed silica, and other ingredients and contents were applied according to Table 3 below, except that the root canal filling material was prepared in the same manner as in Example 1.

Table 1 shows embodiments and comparative examples of the root canal filling compositions of the present invention with different contents of C₃S.

TABLE 1

| | Exam- | Exam- | Comparison | Comparison |
|---|---|---|---|---|
| Ingredients | ple 1 | ple 2 | Example 1 | Example 7 |
| C3S | 25 | 32.4 | 23 | 32.4 |
| | | | | (average particle |
| | | | | size 5 μm) |
| Li2CO3 | 7.2 | 7.2 | 7.2 | 7.2 |
| NaF | 0.5 | 0.9 | 0.5 | 0.9 |
| Fumed SiO2 | 1.8 | 1.8 | 1 | 1.8 |
| CaCl2 | 0.3 | 0.6 | 0.3 | 0.6 |
| ZrO2 | 34.2 | 30.5 | 35 | 30.5 |
| PEG-300 | 30 | 25.4 | 32 | 25.4 |
| H2O | 1 | 1.2 | 1 | 1.2 |
| Total | 100 | 100 | 100 | 100 |
| Cure Time (min) | 240 | 120 | 300 | 130 |
| Compressive | 5 to 6 | 6 to 9 | <2 | 5 to 8 |
| strength (MPa) | | | | |
| Spontaneous healing | ◯ | ◯ | X | ◯ |
| Fluidity (mm) | 29.9 | 28.1 | 35 | 28.2 |

Table 2 shows embodiments and comparative examples of the root canal filling compositions of the present invention with different contents of Li₂CO₃.

TABLE 2

| | | | | Comparison | Comparison | Comparison |
|---|---|---|---|---|---|---|
| Ingredients | Example 3 | Example 4 | Example 5 | Example 2 | Example 3 | Example 4 |
| C₃S | 32.4 | 32.4 | 32.4 | 35 | 35.8 | 31.5 |
| Li₂CO₃ | 5 | 7.2 | 10 | 0 | 3 | 12 |
| NaF | 0.9 | 0.9 | 0.9 | 0.9 | 0.9 | 0.9 |
| Fumed SiO₂ | 1.8 | 1.8 | 1.8 | 0.5 | 0.5 | 1.8 |
| CaCl2 | 0.6 | 0.6 | 0.6 | 0.6 | 0.6 | 0.6 |
| ZrO2 | 31.5 | 30.5 | 29.0 | 32 | 30.5 | 28 |
| PEG | 26.6 | 25.4 | 24.1 | 29 | 26.7 | 24 |
| H2O | 1.2 | 1.2 | 1.2 | 2 | 2 | 1.2 |
| Total | 100 | 100 | 100 | 100 | 100 | 100 |
| Cure Time (min) | 160 | 120 | 100 | 130 | 120 | 110 |
| Compressive | 5 to 6 | 6 to 9 | 8 to 10 | <2 | <3 | 17 to 19 |
| strength (MPa) | | | | | | |
| Spontaneous | ◯ | ◯ | ◯ | X | X | ◯ |
| healing | | | | | | |
| Fluidity (mm) | 29.2 | 28.1 | 27.5 | 31.2 | 30.7 | 23.1 |

Table 3 shows embodiments and comparative examples of the root canal filling compositions of the present invention that vary in the content of pozzolanic reactants (SiO₂). In the present invention, pozzolanic reactants refer to fumed silica, silica fume, or nano-silica.

Furthermore, the difference between Examples 2, 8, and 9 is the different particle size and average particle size of the pozzolanic reactants that are self-healing promoters. Example 2 has a particle size of 200 μm and a particle size of 50 μm, Example 8 has a particle size of 50 μm and a particle size of 8 μm, Example 9 has a particle size of 100 μm and a particle size of 20 μm, and Example 10 has a particle size of 100 μm and a particle size of 10 μm.

TABLE 3

| | | | | | | | Comparison | Comparison |
|---|---|---|---|---|---|---|---|---|
| Ingredients | Example 2 | Example 6 | Example 7 | Example 8 | Example 9 | Example 10 | example 5 | example 6 |
| C3S | 32.4 | 32.7 | 32 | 32.4 | 32.4 | 32.4 | 32.4 | 32.4 |
| Li2CO3 | 7.2 | 7.2 | 7.2 | 7.2 | 7.2 | 7.2 | 7.2 | 7.2 |
| NaF | 0.9 | 0.9 | 0.9 | 0.9 | 0.9 | 0.9 | 0.9 | 0.9 |
| Pozzolanic | 1.8 | 1 | 10 | 1.8 | 1.8 | 1.8 | 0 | 15 |
| Reactants (SiO₂) | | | | | | | | |
| CaCl₂ | 0.6 | 0.3 | 1 | 0.6 | 0.6 | 0.6 | 0.6 | 0.6 |
| ZrO₂ | 30.5 | 30.5 | 27 | 30.5 | 30.5 | 30.5 | 31 | 21 |
| PEG | 25.4 | 26.2 | 20.7 | 25.4 | 25.4 | 25.4 | 26.7 | 21.7 |
| H₂O | 1.2 | 1.2 | 1.2 | 1.2 | 1.2 | 1.2 | 1.2 | 1.2 |
| Total | 100 | 100 | 100 | 100 | 100 | 100 | 100 | 100 |
| Cure Time (min) | 120 | 180 | 110 | 120 | 120 | 120 | 250 | 100 |
| Compressive | 6 to 9 | 5 to 6 | 7 to 10 | 9 to 10 | 7 to 9 | 7 to 9 | <3 | 14 to 16 |
| strength (MPa) | | | | | | | | |
| Spontaneous | ◯ | ◯ | ◯ | ◯ | ◯ | ◯ | X | ◯ |
| healing | | | | | | | | |
| Fluidity (mm) | 28.1 | 25.1 | 28.3 | 28.5 | 28.1 | 28.4 | 23.5 | 35.0 |

Additional note:
The difference between Example 2, Example 8, and Example 9 is due to the different particle size and average particle size of the self-healing promoter.
Example 2: Particle size 200 μm, particle size 50 μm
Example 8: Particle size 50 μm, particle size 8 μm
Example 9: Particle size 100 μm, particle size 20 μm
Example 10: Particle size 100 μm, particle size 10 μm

### Test Results

### Setting Time

Test method: The setting time was evaluated according to ISO 6876:2012 for root canal filling materials. To evaluate the setting time, a plaster mold with an inner diameter of 10 mm and a height of 1 mm was stored at a temperature of 37° C. and a relative humidity of 95% for 24 hours, removed, filled with a sample of root canal filling material, and placed in an oven at a temperature of 37° C. and a relative humidity of 95% for curing. To check the degree of curing, an indenter with a weight of 100 g, a flat end, and a diameter of 2 mm according to ISO 6876:2012 was prepared, and a certain time was set and repeatedly checked until no indentation occurred. The average values obtained by measuring the time from the end of mixing to this point three times are shown in Tables 1 to 3 above.

Results: Referring to FIG. 4 and Tables 1 to 3, it can be seen that the setting time is between 100 and 180 minutes for all embodiments, except for Example 1. This shows that as the content of tricalcium silicate increases, the setting time decreases, the flowability decreases, and the compressive strength increases. On the other hand, when looking at the comparative examples, it was found that Comparative Example 1 with the content of tricalcium silicate below the range was the most delayed. In addition, Comparative Example 8, which has the same ingredients and content as Example 2, has an average particle size of 5 μm and shows similar properties to Example 2, but clogging of the syringe tip by granules occurred. Therefore, among all the components, the chemical component that affects the setting time is tricalcium silicate (C₃S), and the same phenomenon and results were observed for lithium carbonate (Li₂CO₃), sodium fluoride (NaF), and calcium chloride (CaCl₂) as the content of tricalcium silicate increased.

### Compressive Strength

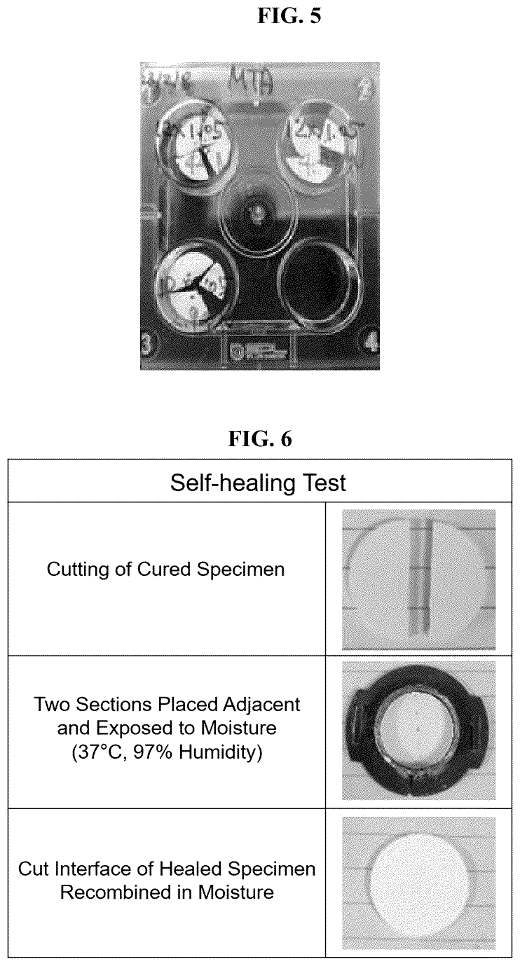

Methods: To evaluate the compressive strength of root canal filling materials, compressive strength specimens were prepared according to ISO 6876:2012 by filling a plaster mold with a diameter of 10 mm and a hole of 1 mm depth and separating them after 3 days of storage in a 37° C. constant temperature and humidity at humidity above 95%. The separated specimens were tested for compressive strength using an Instron type universal testing machine at a speed of 1 mm/min. FIG. 5 is an example of compressive strength, and the average values of five measurements are shown in Tables 1 to 3 above.

Test Results: Referring to FIG. 5 and Tables 1 to 3, it can be seen that all embodiments have a compressive strength of 5 to 10 MPa. However, the compressive strength decreased to less than 5 MPa when the tricalcium silicate was less than the range, contained no or a small amount of lithium carbonate, and contained no fumed silica, while the compressive strength was greater than 10 MPa when containing an excess of lithium carbonate and fumed silica. Thus, it was confirmed that the compressive strength depends on the content of tricalcium silicate (C₃S), lithium carbonate (Li₂CO₃), and pozzolanic reactants (at least one of fumed silica, silica fume, and nano-silica, SiO₂). It was also found that the range of compressive strength (5 to 10 MPa or less) for easy removal was 25 to 35 wt %, 5 to 10 wt %, and 1 to 10 wt % for the contents of tricalcium silicate, lithium carbonate, and fumed silica, respectively. On the other hand, it was found that various property changes are likely to occur when the contents of calcium solubilization inhibitors, curing aids and thickeners are changed simultaneously.

### Self-Healing of Microcracks

Test method: To check the self-healing of microcracks, so-called micro leakage sites, the root canal filling material was sufficiently cured at a temperature of 37° C. and a moisture content of more than 90%, and then separated into two sections as shown in FIG. 6. The two separated sections were kept close together again within 1 mm, maintained at the above temperature and moisture conditions, and if the two sections were not separated by visual observation, they were removed from the moisture and evaluated for self-healing by checking whether they separated again into two sections when impacted. Since the above test method for self-healing has not been defined to date, we evaluated self-healing only by checking the separation after re-curing.

Test Results: Referring to FIG. 6 (test diagram of Example 2) and Tables 1 to 3, it can be observed that all embodiments containing pozzolanic reactants (i.e., at least one of fumed silica, silica fume, or nano-silica) did not separate into two sections when subjected to a repeated separation→maintenance→separation sequence under external impact, as per the test method described above. In contrast, separation was observed under external impact in examples containing tricalcium silicate below the specified range, lacking lithium carbonate and containing only a small amount of fumed silica, containing a small amount of lithium carbonate and a small amount of fumed silica, and lacking fumed silica. These examples showed no self-healing effect.

Specifically, by examining Table 3, which shows the content of the pozzolanic reactants (at least one of fumed silica, silica fume, and nano-silica), it can be seen that examples which have the pozzolanic reactants within the content range of the present invention are capable of self-healing. Furthermore, among the pozzolanic reactants, the embodiment which contained fumed silica and silica fume in a ratio of 1:1 exhibited the best strength within a strength (below 10 MPa) that is easy to remove during treatment, followed by the embodiment which contained the most fumed silica. It was also found that embodiments which contained fumed silica and nano-silica, and nano-silica and silica fume, exhibited almost similar properties.

This showed that the pozzolanic reactants (at least one of fumed silica, silica fume, and nano-silica), when mixed with the tricalcium silicate material and cured, impart a self-healing function that fills the space in the cracks.

### Flowability

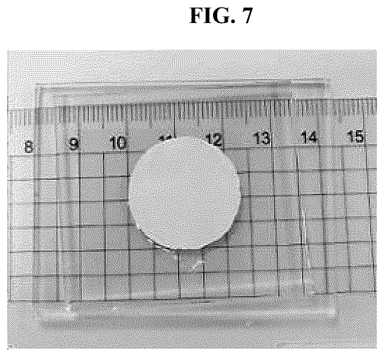

Test method: The flowability evaluation of root canal fillings was performed according to the relevant standard ISO 6876:2001. To evaluate the flowability, 0.05 ml of the root canal filling material was placed in the center of one glass plate (minimum dimensions of 40 mm×40 mm, approximately 5 mm thick) using a syringe capable of injecting 0.05 ml of the sample. After 180 seconds of mixing, a second glass plate was placed on top of the sample center for a total weight of 120 g. Ten minutes after the start of mixing or after placing the sample on the glass plate, the pendulum was removed and the maximum and minimum diameters of the specimen were measured. If the difference between the two diameters was less than 1 mm, the average value was calculated and recorded; if it was more than 1 mm, the test was repeated. FIG. 7 is an example of a diameter measurement, and the average of the results of five measurements is shown in Tables 1 through 3 above.

Results: Referring to FIG. 7 and Tables 1 to 3, it can be seen that the flow rate is between 25 mm and 30 mm in all embodiments. From the results of the embodiments, it can be seen that the flow rate increases with an increase in the content of polyethylene glycol (PEG-300) as a thickener, and the flow rate increases with an increase in the content of pozzolanic reactants (at least one of fumed silica, silica fume, and nano-silica). However, the flowability of the pozzolanic reactants is significantly affected when shear stress is applied, and the flowability decreases when the shear stress is removed. This is presumably due to the networking properties of silica. On the other hand, for the comparison examples, it was found that the fluidity varied depending on the content of the characteristic component.

The above embodiments are exemplary only, and various modifications and other equally valid embodiments will be apparent to a person having ordinary skill in the technical field to which the invention belongs. The true technical scope of the invention will therefore have to be determined by the technical idea of the invention as recited in the claims.

What is claimed is:

1. A dental self-healing root canal filling composition comprising:
a hardener;
a self-healing promoter;
a calcium solubilization inhibitor;
a bioactive agent;
a curing aid;
a radiopaque material;
a thickener; and
purified water.

2. The dental self-healing root canal filling composition of claim 1,
wherein the composition comprises:
25 to 35 wt % of the hardener;
1 to 10 wt % of the self-healing promoter;
0.5 to 1.5 wt % of the calcium solubilization inhibitor;
5 to 10 wt % of the bioactive agent;
0.3 to 1 wt % of the curing aid;
25 to 35 wt % of the radiopaque material;
15 to 40 wt % of the thickener; and
0.5 to 2 wt % of purified water.

3. The dental self-healing root canal filling composition of claim 1,
wherein the hardener comprises tricalcium silicate (C₃S) having a purity of 90 to 98% and an average particle size of 3 μm or less.

4. The dental self-healing root canal filling composition of claim 1,
wherein the self-healing promoter comprises at least one selected from the group consisting of nano-silica (SiO₂), silica fume (SiO₂), and fumed silica (SiO₂).

5. The dental self-healing root canal filling composition of claim 4,
wherein the self-healing promoter has a particle size of 100 nm or less and an average particle size of 10 to 20 nm.

6. The dental self-healing root canal filling composition of claim 1,
wherein the calcium solubilization inhibitor comprises sodium fluoride (NaF), and wherein the bioactive agent comprises lithium carbonate (Li₂CO₃).

7. The dental self-healing root canal filling composition of claim 1,
wherein the curing aid comprises at least one selected from the group consisting of calcium sulfate hemihydrate (CaSO₄·½H₂O), calcium sulfate dihydrate (CaSO₄·2H₂O), calcium chloride (CaCl₂), and calcium formate (Ca(HCOO)₂), and
wherein the radiopaque material comprises at least one selected from the group consisting of zirconium oxide (ZrO₂), tantalum oxide (Ta₂O₅), zinc oxide (ZnO), barium sulfate (BaSO₄), bismuth oxide (Bi₂O₃), barium oxide (BaO), iodoform (CHI₃), and calcium tungstate (CaWO₄).

8. The dental self-healing root canal filling composition of claim 1,
wherein the thickener comprises at least one selected from the group consisting of polyethylene glycol (C₂ₙH₄ₙ₊₂Oₙ₊₁), 1,3-propanediol (CH₂(CH₂OH)₂), and 1,3-butanediol (C₄H₁₀O₂).

9. The dental self-healing root canal filling composition of claim 1,
wherein the composition is used for self-healing, and wherein the composition has (a) a compressive strength of 5 to 10 MPa and (b) a flow rate of 25 to 30 mm.

10. A method for preparing a dental self-healing root canal filling material, the method comprising:
(a) preparing a hardener comprising tricalcium silicate (C₃S);
(b) mixing 25 to 35 wt % of tricalcium silicate (C₃S), 1 to 10 wt % of a self-healing promoter, 0.5 to 1.5 wt % of a calcium solubilization inhibitor, 5 to 10 wt % of a bioactive agent, 0.3 to 1 wt % of a curing aid, 25 to 35 wt % of a radiopaque material, 15 to 40 wt % of a thickener, and 0.5 to 2 wt % of purified water to prepare a mixture;
(c) maintaining the mixture under a vacuum of −0.1 MPa or less for 15 to 20 minutes to eliminate bubbles and increase filling density;
wherein the self-healing promoter comprises at least one selected from the group consisting of nano-silica (SiO₂), silica fume (SiO₂), and fumed silica (SiO₂).

11. The method of claim 10,
wherein the calcium solubilization inhibitor comprises sodium fluoride (NaF), the bioactive agent comprises lithium carbonate (Li₂CO₃),
the curing aid comprises at least one selected from the group consisting of calcium sulfate hemihydrate (CaSO₄·½H₂O), calcium sulfate dihydrate (CaSO₄·2H₂O), calcium chloride (CaCl₂), and calcium formate (Ca(HCOO)₂),
the radiopaque material comprises at least one selected from the group consisting of zirconium oxide (ZrO₂), tantalum oxide (Ta₂O₅), zinc oxide (ZnO), barium sulfate (BaSO₄), bismuth oxide (Bi₂O₃), barium oxide (BaO), iodoform (CHI₃), and calcium tungstate (CaWO₄), and
the thickener comprises at least one selected from the group consisting of polyethylene glycol (C₂ₙH₄ₙ₊₂Oₙ₊₁), 1,3-propanediol (CH₂(CH₂OH)₂), and 1,3-butanediol (C₄H₁₀O₂).

12. The method of claim 10,
wherein step (a) comprises:
(i) preparing a mixture-1 by mixing calcium carbonate (CaCO₃) and silica (SiO₂) in a 3:1 molar ratio with 250 to 350 cc of water;
(ii) mixing the mixture-1 in a zirconia container for 4 to 5 hours using a ball mill to obtain mixture-2;
(iii) pouring mixture-2 into silicone molds and drying using a heating plate;
(iv) heat treating the dried mixture-2 in an electric furnace at 1,350 to 1,600° C. for 5 to 6 hours to calcine and then quenching;
(v) mixing and grinding the quenched mixture-2 using a ball mill for 24 to 30 hours; and
(vi) pulverizing the mixture-2 using a sieve to obtain tricalcium silicate having an average particle size of 3 μm or less.
